(19) United States
(12) Patent Application Publication
(10) Pub. No.: US 2022/0313858 A1
(43) Pub. Date: Oct. 6, 2022

(54) HORSE BEDDING RECYCLING SYSTEM

(71) Applicant: H.H. Sheikha Alyazia Bint Sultan Bin Khalifa Bin Zayed Al Nahyan, Abu Dhabi (AE)

(72) Inventor: H.H. Sheikha Alyazia Bint Sultan Bin Khalifa Bin Zayed Al Nahyan, Abu Dhabi (AE)

(21) Appl. No.: 17/656,381

(22) Filed: Mar. 24, 2022

(30) Foreign Application Priority Data
Mar. 30, 2021 (GB) 2104520.8

(51) Int. Cl. A61L 2/26; A61L 2/10; A61L 2/04; A01K 1/01

(52) U.S. Cl. CPC A61L 2/26; A61L 2/10; A61L 2/04; A01K 1/01; A61L 2202/11

(57) ABSTRACT

A device suitable for recycling horse bedding, the device including a shaker table with a receiving surface arranged to receive the bedding; a duct having an inlet and an outlet; a first airflow generator arranged to lift bedding elements from the horse bedding upwards towards the duct inlet; a second airflow generator arranged to transport the bedding elements within the duct, the duct being shaped to define an inlet transportation region in which the second airflow causes the bedding elements to be drawn from the duct inlet, along a portion of the duct; a suspension region in which the second airflow causes the bedding elements to be suspended within the suspension region of the duct; and an outlet transportation region in which, in the absence of the second airflow, the bedding elements fall due to gravity towards the duct outlet; and one or more UV light sources arranged within the duct to emit UV radiation into the suspension region.

### RELATED APPLICATIONS

This application claims priority to Great Britain Patent Application No. 2104520.8, filed Mar. 30, 2021, the entire content of which is incorporated herein by reference.

### BACKGROUND OF THE INVENTION

Animal bedding is provided in places such as stables or other resting structures to support an animal's body weight when resting. Animal bedding also serves to absorb animal waste, thereby reducing contamination.

Animal bedding can generally be split into animal bedding for small animals, such as rabbits, rodents and the like, and bedding for large animals such as horses, cattle, sheep and the like. Generally, bedding elements such as wood chips or shaving, pieces or straw or the like within bedding for large animals will be larger in size in comprising to those found in bedding for small animals. Moreover, much larger quantities are used.

Animal bedding must be replaced or sanitised after a period of use. Typically, used bedding is removed and replaced daily. However, the replacement of the bedding can be expensive and have a significant environmental impact, particularly bedding for large animals.

It can therefore be desirable to sanitise animal bedding so that the sanitised bedding can be reused.

Current methods of bedding sanitisation utilise a chlorinated water tank or other liquid sanitisation methods. However, the present inventors have identified that such methods are complex, time consuming and can require equipment which occupies a large working volume.

### SUMMARY OF THE INVENTION

According to a first aspect of the present invention there is provided a device for recycling animal bedding such as horse, cattle and/or sheep bedding, the device comprising: a shaker table with a receiving surface arranged to receive the bedding; a duct having an inlet and an outlet; a first airflow generator arranged to lift bedding elements from the bedding upwards towards the duct inlet; a second airflow generator arranged to transport the bedding elements within the duct, the duct being shaped to define: an inlet transportation region in which the second airflow causes the bedding elements to be drawn from the duct inlet, along a portion of the duct; a suspension region in which the second airflow causes the bedding elements to be suspended within the suspension region of the duct; and an outlet transportation region in which, in the absence of the second airflow, the bedding elements fall due to gravity towards the duct outlet; and one or more UV light sources arranged within the duct to emit UV radiation into the suspension region.

Thus, the recycling device according to the first aspect allows used animal bedding to be separated into relatively heavy waste, which is left on the shaker table, and relatively light bedding elements, which are lifted from the shaker table by the first airflow towards the sanitisation duct. By separating the recyclable material, the arrangement allows for the bedding elements to be treated separately from the waste, allowing UV lights within the duct to focus their energy on the relatively lightly soiled bedding elements. The duct is arranged so that the second airflow is caused to move in a manner which creates a swirling air mix flow that causes the bedding elements to move and collide while being generally suspended within a suspension region of the duct, which increases the likelihood outer surfaces of the bedding elements being exposed the UV light. The duct has an outlet underneath the suspension regions so that the sanitised bedding elements can fall by way of gravity from the suspension region to the outlet when the second airflow is turned off. This arrangement provides a quick, easy way of sanitising animal bedding. Also, since the bedding elements are not being sanitised by a liquid, the time taken to perform a sanitisation cycle can be reduced in comparison to know methods.

The duct can comprise a first vertical portion and a second vertical portion in fluid communication via a bridging portion, the first and second vertical portions being laterally offset by at least the diameter of the second vertical portion. The walls of the first and second vertical portions can be more vertical than horizontal; for example, more parallel than orthogonal relative to the axis of rotation of the first fan.

As the bedding elements can fall directly back into the separated waste when the second airflow is removed, it is advantageous that the bedding elements are lifted and linearly transported. This allows for a compact arrangement while providing an efficient system. The first and second vertical portions can be linear and connected with a linear bridge portion which extends orthogonally with respect to the first and second vertical portions.

The duct inlet can be at the base of the first vertical portion and the duct outlet can be at the base of the second vertical portion. This provides an efficient and compact arrangement. As the sanitised bedding elements can fall under their own weight to exit the duct, the need for active extraction of the sanitised bedding elements within the second airflow is reduced. The arrangement can be compact as the inlet and outlet can be of the same cross sectional area of the first and second vertical duct portions, respectively. The suspension region can comprise a mesh panel which extends from an inner roof surface of the passage region of the duct towards the duct outlet at an angle which is skew with respect to the longitudinal axis of the second vertical portion, the mesh being of a coarseness which permits at least some of the second airflow air to pass through it but substantially does not permit bedding elements to pass through it. The mesh can for example defines holes which are no larger than 15 mm in diameter and in some embodiments can be approximately 10 mm in diameter or less.

This arrangement forces the bedding elements to follow a “U” shaped path around the mesh within the second vertical portion or suspension region. This can increase the UV exposure to the individual bedding elements while maintaining a compact design. The mesh panel can allow the bedding elements to move within a turbulent airflow so that the individual elements are regularly changing orientation which can increase the surface area exposed to UV light. The mesh panel can extend into a plane of an inner base surface of the transportation or suspension region of the duct. The mesh panel can be angled between 20 and 60 degrees with respect to the longitudinal axis of the first and second vertical portions, as this can help to direct incoming airflow down the U shaped path.

The first and second vertical regions can be arranged with their longitudinal axes in parallel and the base of the first vertical portion is in line with or vertically higher than the base of the second vertical portion. This arrangement allows for a compact design whilst also enabling used bedding to be loaded into and unloaded out of the device at approximately similar heights. Alternatively, the first and second vertical regions may not be aligned along their longitudinal axis but instead arranged on an angle.

The first outlet can be covered with a removable tray for supporting bedding elements that fall to the outlet and the duct further comprises a second outlet located above the first outlet, the second outlet being covered by a second mesh, the second mesh being of a coarseness which permits at least some of the second airflow air to pass through it and out of the duct but substantially does not permit bedding elements to pass through it.

This arrangement is advantageous as it prevents pressure building within the duct that could then force bedding elements away from the UV lights or damage the device. The removable tray may include a mesh, which advantageously allows sand, dust and the like to fall through it but does not allow passage of sanitised bedding elements; the mesh can for example defines holes which are no larger than 15 mm in diameter and in some embodiments can be approximately 10 mm in diameter or less.

A heat source can be arranged at the duct opening to heat bedding elements entering the duct. The heat source can be angled across the duct inlet. The heat source can provide a hot, dry airflow. Bedding elements can be damp when first separated from waste in the used bedding and therefore it can be advantageous to use a heated airflow to assist in drying. The heat source can also provide an initial level of sanitisation to the bedding elements.

The duct inlet can include a radially flared hood which tapers inwardly towards the duct to direct bedding elements lifted by the first airflow into the duct. This is advantageous as it provides a channel for the bedding elements.

A second heat source can be arranged to provide heat to the removable tray. The second heat source can provide dry air to the bedding elements which have fallen into the removable tray. This is advantageous as the heat source can cause the bedding elements in the removable tray to be lifted and then fall back into the tray to keep the bedding elements moving.

At least one of the UV light sources can be arranged to illuminate the removable tray. This is advantageous as it provides further sanitisation to bedding elements which have passed through the device while the device cycles through further bedding elements.

The UV light sources can be UVC. This is advantageous as it provides an efficient source of antiviral and antibacterial light.

The receiving surface can be formed from a mesh and the first airflow generator is arranged on the opposite side of the receiving surface relative to the duct such that it blows the first airflow through the receiving surface.

This arrangement allows the bedding elements to be lifted into the duct without the bedding elements becoming stuck in the first airflow equipment.

The first airflow generator can be distinct from the second airflow generator. The first airflow can be greater than the second airflow due to the first fan being arranged to generate a greater airflow than the second fan.

The first airflow generator can comprise a first fan arranged on an opposite side of the receiving surface of the shaker table and arranged to blow through the receiving surface to force bedding elements towards the duct inlet.

The first fan can be configured to provide a first airflow of at least 500 cubic meters per minute, more preferably at least 1,000 cubic meters per minute, more preferably at least 1,500 cubic meters per minute and in some embodiments approximately 1,700 cubic meters per minute. It is preferred that the first fan does not generate more than 2,500 cubic meters per minute so as to be selective in terms of separating bedding elements from animal waste.

The second airflow generator can comprise a second fan arranged within the duct closer to the inlet than the outlet and arranged to draw air from the inlet and force it along the duct.

The second fan can be configured to provide a first airflow of at least 350 cubic meters per minute, more preferably at least 750 cubic meters per minute, more preferably at least 1,000 cubic meters per minute and in some embodiments approximately 1,200 cubic meters per minute. It is preferred that the second fan does not generate more than 1,800 cubic meters per minute so as to manage air pressure within the system.

In one embodiment the bedding is horse bedding. Used horse bedding can comprise waste and bedding material. An example of such bedding material can be wood chips, straw, or pellets.

According to a second aspect of the present invention there is provided a method for recycling horse bedding, the method comprising, shaking a receiving surface arranged to receive the horse bedding; generating a first airflow to lift bedding elements from the horse upwards towards a duct inlet, the duct having an outlet; generating a second airflow arranged to transport the bedding elements within the duct, the duct being shaped to define: an inlet transportation region in which the second airflow causes the bedding elements to be drawn from the duct inlet, along a portion of the duct; a suspension region in which the second airflow causes the bedding elements to be suspended within the suspension region of the duct; and an outlet transportation region in which, in the absence of the second airflow, the bedding elements fall due to gravity towards the duct outlet; and emitting UV radiation into the suspension region.

This arrangement provides an efficient method for separating and sanitising bedding elements from used animal bedding in order for the bedding elements to be reused.

The first and second airflows can be stopped to allow the sanitised bedding elements to fall towards the outlet.

The first and second airflows can be generated for a first time period and stopped for a second time period. The first time period can be 1 minute, the second time period can be 40 seconds, with these periods alternating for a 15 minute cycle upon activation of the device.

### BRIEF DESCRIPTION OF THE DRAWINGS

Embodiments of the invention will now be described, by way of example only, with reference to the accompanying drawings, of which:

FIG. 1 is a schematic drawing of recycling system according to an embodiment of the invention; and

FIG. 2 is a schematic diagram of a method of using the recycling system according to an embodiment of the invention.

### DETAILED DESCRIPTION OF THE DRAWINGS

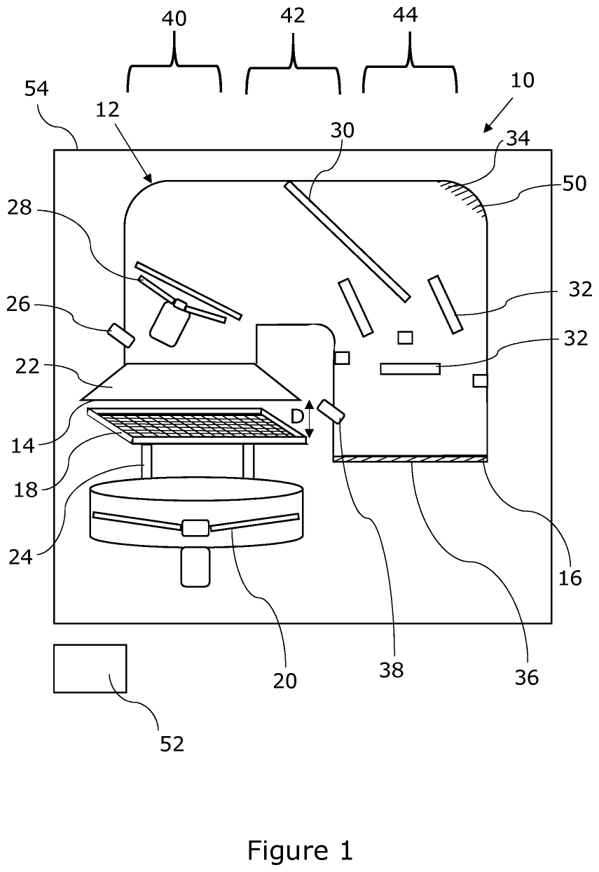

FIG. 1 is a diagram of a recycling device 10 according to a first embodiment of the invention. The device 10 is arranged to sanitise dirty animal bedding, such as horse bedding, for subsequent reuse. As will be appreciated, dirty animal bedding includes bedding elements such as wood chips, pellets, or straw and can also contain liquid and/or solid animal waste.

The device 10 includes a shaker table 18 defining a tray for receiving used animal bedding. The table 18 is arranged with at least one actuator 24 which, when switched on, causes the table 18 to oscillate. This agitation can result in bedding elements with different masses separating.

A first airflow generation device is provided to lift bedding elements from the shaker table 18 upwards towards an inlet of a duct 12. In the illustrated embodiment, the first airflow generation device is a fan 20 arranged on the opposite side of the shaker table 18 with respect to the duct 12 so that bedding elements are blown upwards from the shaker table 18 towards the duct inlet. The shaker table 18 is formed from a mesh in order to allow the first airflow to pass through the shaker table 18 and impact the underside of the used bedding. The fan 20 can for example have swept diameter of 125 cm and generate an air output of 60,000 CFM (approximately 1,700 cubic meters per minute). In other embodiments, the fan 20 can generate an air output of between 500 cubic meters per minute and 2,500 cubic meters per minute for example.

The duct 12 comprises an inlet 14 for dirty bedding elements to enter the duct 12 and an outlet 16 for sanitised bedding elements to exit the duct 12.

In the illustrated embodiment, the duct 12 has an inverted U shape, with a first vertical portion 40 coupled to a second vertical portion 44 via a generally horizontal bridging portion 42. The bridging portion 42 provides fluid communication between the tops of the first and second vertical portions 40, 42. The cross sectional area of the duct and its profile can vary depending on size of the load, space available, and end user choice. In this embodiment, the duct has a rectangular profile of width and height being roughly 1 m. In other embodiments the duct can have any suitable profile. The cross sectional area of the duct 12 can be constant along the duct 12. The duct 12 can for formed from any suitable material, for example a metal or hard plastics material.

The base 22 of the first vertical portion 42 defines the duct inlet and in this embodiment is flared to define a collecting hood or shroud which tapers inwardly so as, in use, to channel the bedding elements into the duct 12. The first vertical portion 40 can be situated directly above the shaker table 18, and preferably have their central axes aligned, with the duct inlet 14 being axially spaced from the shaker table by a distance D, which can for example be 80 cm. As will be described in more detail, the distance D provides a handover region where lifted bedding elements can be passed from the first airflow to a second airflow as they are acted on by the first airflow and the second airflow.

A second airflow generator is arranged within the duct 12 to force the bedding elements to move along the duct 12. In this embodiment the second airflow generator is a fan 28 mounted in a generally central position within of the first vertical portion 40a of the duct 12 and angled slightly towards the bridging portion 42 of the duct 12. The fan 28 can for example have swept diameter of 75 cm and generate an air output of 42,000 CFM (approximately 1,200 cubic meters per minute) The fan 28 is located close enough to the duct inlet 14 to define a handover region at the duct inlet 14 where bedding elements are drawn into the inlet 14 by the fan 28, but in other embodiments the first fan 20 can be sized and powered to push the bedding elements into the duct inlet 14.

The second airflow transports the bedding elements through the bridging portion 42 to the second vertical portion 44. In the illustrated embodiment, the duct outlet 16 is located at the base of the second vertical portion 44 and closed by a removable collection tray 36. An upper corner of the second vertical portion 44 which is furthest from the inlet 14 defines a vent 34 provided with a mesh cover that spans the vent. A mesh deflection panel 30 extends from an inner duct surface within the bridging portion 42 into the second vertical portion 44. The mesh deflection panel 30 can be generally planar and extend the full width of the duct 12 and extend lengthwise to terminate in a location that almost reaches a plane of the inner base surface of the bridging portion 42. The mesh deflection panel 30 can be orientated at a skew angle with respect to the longitudinal axes of the bridging portion 42 and second vertical portion 44; for example, the mesh panel 30 can be angled between 20 and 60 degrees with respect to the longitudinal axis of the second vertical portion 44.

The mesh cover and mesh deflection panel 30 can each be formed from mesh defining spaces of approximately 10 mm in diameter. Thus, the mesh in each case allows at least some of the second airflow to pass through the mesh but is fine enough to inhibit the passage of the bedding elements.

Consequently, at least some of the second airflow follows a generally inverted S shaped path through the duct 12 moving up from the inlet 14, past the second fan 28 before bending around and down, following the mesh deflection panel 30 before turning back on itself upwardly around the far side of the mesh deflection panel 30 towards the vent 34 due to the outlet 16 being at least partially by the collection tray 36. The second vertical portion 44 of the duct 12 is therefore partitioned into a generally U shaped path by the mesh deflection panel 30. As the second airflow moves up the right hand side of the U shaped path, some of the second airflow can exit the duct via the vent 34 and some can pass to the left through the mesh deflection panel 30 to merge with incoming airflow and travel back down the U. The angled mesh deflection panel 30 inhibits UV sanitised bedding elements from moving back into the inlet transportation region, while the meshed vent 34 inhibits bedding elements leaving the duct via the vent. The second airflow can therefore move in a swirling and/or turbulent manner that defines an air mix suspension region in which bedding elements move in random and/or fluttering patterns within the second vertical portion 44 of the duct.

The suspension region comprises at least one UV light source 32 arranged to illuminate at least a portion of the suspension region. The UV light source 32 can be a series of lights, such as UVC sources which provide a series of light curtains or general illumination. In the illustrated embodiment, nine UV light sources are provided to direct UV light into and across the suspension region from different positions and at different orientations so as to irradiate some or all of the suspension region; namely, three lights mounted on each lateral side wall facing the opposite lateral sidewall in a generally U shaped arrangement, one mounted on a sidewall closest to the inlet and facing up to direct light towards incoming bedding elements, a further light mounted on the opposite sidewall facing up to direct light as bedding elements moving upwardly in the suspension region, and a light mounted between the latter two and facing downwards to direct light towards bedding elements on or adjacent to the collection tray. Each UV light can for example comprise an 80 W UVC strip light. In other embodiments, any suitable light configuration can be provided that can perform at least some sanitisation of bedding elements moving within the suspension region.

It will be appreciated that despite being referred to as a suspension region, the bedding elements are not strictly suspended stationary in the duct 12 but are instead suspended within the suspension region by the second airflow. This suspension allows the bedding elements to continuously move or flutter in a mixing manner while being illuminated by the UV light sources 32.

When the second airflow is ceased, the suspended bedding elements will fall under gravity to the lowest point of the second vertical portion 44, out of the duct outlet 16 and can therefore be caught by the collection tray 36. The collection tray 36 can be removed to allow the bedding elements to be collected by a wheel barrow for example, located under the outlet 16.

Thus, as will be appreciated from the foregoing description, duct 12 is shaped to define: an inlet transportation region in which the second airflow causes the bedding elements to be drawn from the duct inlet, along a portion of the duct; a suspension region in which the second airflow causes the bedding elements to be suspended within the suspension region of the duct; and an outlet transportation region in which, in the absence of the second airflow, the bedding elements fall due to gravity towards the duct outlet.

The collection tray 36 can be arranged such that a further UV light or UV light from the suspension region illuminates any bedding elements landed on the tray 36. This can provides further sanitisation. The collection tray can comprise a mesh of approximately 10 mm.

An optional heat source 26 can be provided within the first vertical portion 40 to heat the bedding elements either before or after they enter the duct 12. The heat source 26 can be angled across the duct inlet 14 or first vertical portion 40.

The second vertical portion 44 can comprise a second heat source 38 to provide a flow of air to the removable tray 36. This heated air can cause the collected bedding elements to stay in motion within the outlet transportation region to further increase sanitisation.

The recycling device also includes a controller 52 which is electrically coupled to the actuator(s) 24, fans 20, 28 and the heaters 26, 38 to control operation of them. The controller can be any suitable computing device having a control input such as a button or other electrical signal generator. The controller can be programmed with one or more control programs to control operation of some or all of the above-mentioned components of the device 10. A user can use the button to select or activate a control program, or a particular component.

Components of device 10 can be encased within a body 54 having an inlet opening for enabling a user to place dirty bedding on the shaker table 18 and a collection opening for receiving a wheelbarrow or the like to be placed under the outlet 16. The inlet opening can be provided with a door or hatch (not shown) which can be closed while device 10 is operating, in which case it is preferred that the shaker table 18 is enclosed at its sides and rear to that the door or hatch, when closed, defines a passage with the side walls and rear wall between the shaker table 18 and the duct inlet 14.

While in the illustrated embodiment the first and second airflows are defined by distinct fans, in other embodiments a single fan can define both airflows.

Moreover, while the duct 12 in the illustrated embodiment is generally an inverted U shape, in other embodiments the duct can take any suitable shape and may not include features such as the mesh covered vent 34 and/or the collection tray 36 and/or the mesh deflection panel 30. In one example, the duct second vertical portion can extend upwardly from the bridging portion so as to have a generally side on, inverted S shape, with the outlet and removable collection tray being at the base of second vertical portion at a level adjacent the base of the bridge portion; the illustrated embodiment is however advantageous over such an arrangement due to providing a more compact arrangement with a smaller envelope.

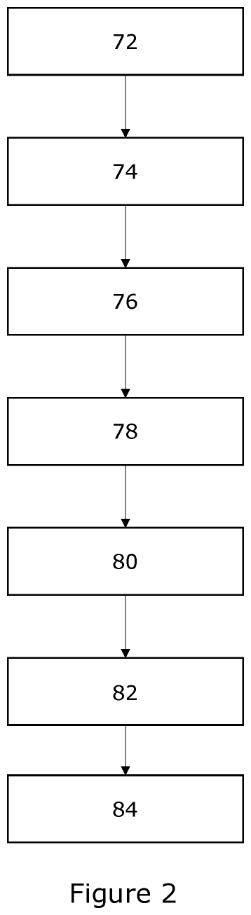

FIG. 2 shows an exemplary method of operating the device shown in FIG. 1. At step 72, used animal bedding is placed onto the shaker table 18 of the device 10.

With a programmed setting, the device can be initiated which actuates the shaker table 74 and generates the first airflow 76. As the bedding elements are lifted by the first airflow into the first vertical portion, the second airflow can be initiated. The second airflow can be generated 78 at the same time as the first airflow is generated or can be delayed to allow for bedding elements to reach the handover region above the shaker table. Heat can be applied as the second airflow is generated 78 so that either the bedding elements are heated before entering the second airflow or the second airflow is itself heated.

The second airflow provides a transport means to move the elements from the first vertical portion to the bridging portion and onto the second vertical portion. The geometry of the duct can be optimised to increase the amount of time the bedding elements are moving within the airflow and thus suspended. UV illumination is provided 80 within the suspension region sanitises the suspended bedding elements. As it is advantageous to keep the bedding elements individually moving and rotating, the duct can be provided with means of extending the airflow route, for example by providing a mesh plate to divert the bedding elements.

After the bedding elements have been suspended or circulating within the UV illuminated region, the second airflow can be switched off 82 to allow the bedding elements to fall under gravity. The bedding elements can fall into a removable tray at the lowest point of the second vertical element which can be manually emptied to retrieve the sanitised bedding elements. A further heated airflow can be provided 84 to the tray area to keep the fallen bedding elements mobile. UV illumination can be provided to the tray area to provide further sanitisation of the bedding elements.

The method can cycle through the steps 76 to 82. In this embodiment, the airflows are generated 76, 78 for a period of time, causing the bedding elements to move via the airflows in the suspension region 80. The airflows are then terminated 82 for a period of time, allowing the suspended bedding elements to fall 84. The cycle can then repeat with the airflows being further generated 76, 78 so that another batch of bedding elements are lifted from the shaker table and suspended under UV illumination 80. The airflows can then be again switched off 82, allowing the second batch of sanitised bedding elements to fall out of the airflow 84.

The method can be carried out in isolated batches by depositing additional used bedding onto the shaker table before a further cycle is run. Alternatively, the method can be carried out so that the shaker table receives the used bedding and the airflow cycles between switching on and off over a predetermined time period. In this embodiment, a portion of bedding elements in the shaker table would be lifted. Bedding elements which are closer to the top of the pile on the shaker table or elements which are less damp and thus lighter may be lifted in earlier cycles. Once the first cycle is deposited in the receiving tray, the second cycle can begin. The second cycle can then be collected into the removable tray and a further cycle can begin. After a predetermined time period has passed, the removable tray can be released to allow multiple cycles of sanitised bedding elements to be collected.

In one example, the airflows can be generated for 1 minute, followed by a 40 second interval of reduced or no airflow. This cycle can continue for a time set by a user, for example 15 minutes. The user could thus begin a 15 minute timer and allow the device to automatically run the cycles. When the 15 minute timer expires, the removable tray can be emptied into a collection device such as a wheelbarrow.

It should be noted that the above-mentioned embodiments illustrate rather than limit the invention, and that those skilled in the art will be capable of designing many alternative embodiments without departing from the scope of the invention as defined by the appended claims. In the claims, any reference signs placed in parenthesis shall not be construed as limiting the claims. The word “comprising” can mean “including” or “consisting of” and therefore does not exclude the presence of elements or steps other than those listed in any claim or the specification as a whole. The word “coupled” can mean “attached” or “connected”. The singular reference of an element does not exclude the plural reference of such elements and vice-versa. In an apparatus claim enumerating several parts, several of these parts may be embodied by one and the same item of hardware. The mere fact that certain measures are recited in mutually different dependent claims does not indicate that a combination of these measures cannot be used to advantage.

1. A device for recycling horse bedding, the device comprising:
a shaker table with a receiving surface arranged to receive the bedding;
a duct having an inlet and an outlet;
a first airflow generator arranged to generate a first airflow to lift bedding elements from the bedding upwards towards the duct inlet;
a second airflow generator arranged to generate a second airflow to transport the bedding elements within the duct, wherein the duct is shaped to define:
an inlet transportation region in which the second airflow causes the bedding elements to be drawn from the duct inlet, along a portion of the duct;
a suspension region in which the second airflow causes the bedding elements to be suspended within the suspension region of the duct; and
an outlet transportation region in which, in the absence of the second airflow, the bedding elements fall due to gravity towards the duct outlet; and
one or more UV light sources arranged within the duct to emit UV radiation into the suspension region.

2. The device according to claim 1, wherein the duct comprises a first vertical portion and a second vertical portion in fluid communication via a bridging portion, wherein the first and second vertical portions are laterally offset by at least a diameter of the second vertical portion.

3. The device according to claim 2, wherein the duct inlet is at the base of the first vertical portion and the duct outlet is at the base of the second vertical portion.

4. The device according to claim 2, wherein the suspension region comprises a mesh panel which extends from an inner roof surface of the passage region of the duct towards the duct outlet at an angle which is skew with respect to the longitudinal axis of the second vertical portion.

5. The device according to claim 2, wherein the first and second vertical regions are arranged with their longitudinal axes in parallel and the base of the first vertical portion is in line with, or vertically higher than, the base of the second vertical portion.

6. The device according to claim 1, wherein the outlet is covered with a removable tray for supporting bedding elements that fall to the outlet and the duct further comprises a second outlet located above the first outlet, wherein the second outlet is covered by a second mesh.

7. The device according to claim 1, further comprising a heat source arranged at the duct opening to heat bedding elements entering the duct.

8. The device according to claim 1, wherein the duct inlet includes a radially flared hood which tapers inwardly towards the duct to direct bedding elements lifted by the first airflow into the duct.

9. The device according to claim 1, further comprising a second heat source arranged to provide heat to the removable tray.

10. The device according to claim 1, wherein at least one of the UV light sources is arranged to illuminate the removable tray.

11. The device according to claim 1, wherein the UV light sources are UVC.

12. The device according to claim 1, wherein the receiving surface is formed from a mesh and the first airflow generator is arranged on the opposite side of the receiving surface relative to the duct such that it blows the first airflow through the receiving surface.

13. A method for recycling animal bedding, the method comprising,
shaking a receiving surface arranged to receive the bedding;
generating a first airflow using a first airflow generator to lift bedding elements from the bedding upwards towards a duct inlet, the duct having an outlet;
generating a second airflow using a second airflow generator arranged to transport the bedding elements within the duct, the duct being shaped to define:
an inlet transportation region in which the second airflow causes the bedding elements to be drawn from the duct inlet, along a portion of the duct;
a suspension region in which the second airflow causes the bedding elements to be suspended within the suspension region of the duct; and
an outlet transportation region in which, in the absence of the second airflow, the bedding elements fall due to gravity towards the duct outlet; and
emitting UV radiation into the suspension region.

14. The method according to claim 13, further comprising stopping the first and second airflows to allow the bedding elements to fall towards the outlet.

15. The method according to claim 13, further comprising alternating between generating the first and second airflows for a first time period and stopping the first and second airflows for a second time period.
